(19) United States
(12) Patent Application Publication
(10) Pub. No.: US 2015/0225801 A1
(43) Pub. Date: Aug. 13, 2015

(54) RECORDING AND MAPPING LINEAGE INFORMATION AND MOLECULAR EVENTS IN INDIVIDUAL CELLS

(71) Applicant: California Institute of Technology, Pasadena, CA (US)

(72) Inventors: Long CAI, Pasadena, CA (US); Michael B. ELOWITZ, Pasadena, CA (US); James D. LINTON, Pasadena, CA (US); Joonhyuk CHOI, Pasadena, CA (US); Kirsten L. FRIEDA, Pasadena, CA (US); Sahand HORMOZ, Pasadena, CA (US); Ke-Huan Kuo CHOW, Pasadena, CA (US)

(21) Appl. No.: 14/620,133

(22) Filed: Feb. 11, 2015

Related U.S. Application Data
(60) Provisional application No. 61/938,490, filed on Feb. 11, 2014.

(51) Int. Cl. C12Q 1/68

(52) U.S. Cl. CPC C12Q 1/6888; C12Q 2600/16; C12Q 2600/156

(57) ABSTRACT

Methods and systems for recording and mapping lineage information and molecular events in individual cells are provided. Molecular changes, which may result from random or specific molecular events, are introduced to defined regions in cells over multiple cell cycle generations. Techniques such as fluorescent imaging are applied to track and identify the molecular changes before such information is used for lineage analysis or for identifying key processes and key players in cellular pathways.

### CROSS-REFERENCE TO RELATED APPLICATIONS

This application claims priority from U.S. Provisional Patent Application No. 61/938,490, filed on Feb. 11, 2014, which is incorporated herein by reference in its entirety.

### FIELD OF THE INVENTION

The invention disclosed herein generally relates to methods and systems for creating or triggering molecular changes (e.g., genetic mutations or modification) in defined regions in a genome. In particular, the invention disclosed herein relates to the design and characteristics of such defined regions and methods and systems for creating or triggering molecular changes that lead to or result from certain random or specific molecular events such as signal transduction. Further, the invention disclosed herein relates to methods and systems for capturing, characterizing and analyzing the molecular changes, in order to extrapolate lineage or phylogenetic information connecting such molecular events or record the history of cellular events.

### BACKGROUND

A fundamental problem throughout developmental biology is determining the lineages through which cells differentiate to form tissues and organs. Lineage information is critical for addressing basic developmental questions in diverse systems including the brain and tumor genesis. Although the lineage map of embryonic development in C. elegans was worked out three decades ago (1), systematic techniques that can produce such comprehensive maps in more complex organisms are lacking. Furthermore, in order to understand how lineages are determined, the lineage tree needs to be connected directly to the molecular changes and eventually molecular events that occur in cells to determine developmental decisions.

Existing lineage determination approaches have severe limitations. Most current approaches are based on marking the descendants of selected cells (2, 3). Site-specific recombinases such as FLP and Cre can be used to mark the descendants of particular cells (4-6). More sophisticated variants, such as Brainbow (7), can mark many distinct cells at one time to follow their descendants. However, these techniques do not allow one to follow multiple lineage decisions or reconstruct an entire tree in a single experiment. Finally, no existing technique enables one to systematically record the molecular events that occur during lineage determination within the cells themselves.

What is needed in the art are vastly improved tools for tracking lineage information, capturing molecular changes during development and reading out this information with minimal perturbations to cells and organisms, ideally within the cells themselves.

### SUMMARY OF THE INVENTION

In one aspect, provided herein is a method for characterizing lineage information or recording molecular events among cells in a cell population. The method comprises the steps of: introducing, over a time period of multiple cell cycle generations, a plurality of molecular changes in at least one of one or more genetic scratchpads in one or more cells in a cell population, characterizing, at one or more time points during the time period, a status of molecular changes at each time for the plurality of target sites in each genetic scratchpad in cells in the cell population, wherein the cells are essentially intact or undisrupted, wherein at least one time point in the one or more time points is two or more cell cycle generations from the beginning of the time period; and establishing lineage connections between cells from different cell cycle generations by comparing statuses of molecular changes of the cells.

In some embodiments, the cell population comprises cells that have developed for one or more cell cycle generations. In some embodiments, each genetic scratchpad in the one or more genetic scratchpads comprises a polynucleotide sequence and a plurality of target sites within the polynucleotide sequence. In some embodiments, each of the plurality of mutations is associated with a target site among the plurality of target sites. In some embodiments, the molecular changes represent one or more molecular events: they are either the cause or result of one or more molecular events.

In some embodiments, characterizing step further comprises the steps of applying a set of probes to the cell population and characterizing the mutation status in a plurality of cells in the cell population by detecting the presence or absence of visible signals in the plurality of cells.

In some embodiments, each probe in the set recognizes and binds to a corresponding target sequence in a target site among the plurality of target sites.

In some embodiments, each probe comprises a label that produces a visible signal upon binding between the probe and its unique target sequence.

In some embodiments, each target site comprises a guide sequence that is recognized by a unique guide molecule, and wherein binding of the unique guide molecule to the guide sequence recruits a molecule that is capable of creating a mutation at the target site.

In some embodiments, the guide sequence comprises a nucleotide sequence having a length between about 15 nucleic acids to about 80 nucleic acids. In some embodiments, the guide sequence comprises a nucleotide sequence having a length between about 15 nucleic acids to about 30 nucleic acids.

In some embodiments, the unique guide molecule is a guide RNA (gRNA).

In some embodiments, the molecule is a nuclease, recombinase or integrase. In some embodiments, the nuclease is Cas9 nuclease

In some embodiments, the multiple time points during the time period cover two or more cell cycle generations. In some embodiments, the multiple time points during the time period cover three or more cell cycle generations. In some embodiments, the multiple time points during the time period cover five or more cell cycle generations.

In some embodiments, the plurality of molecular changes comprises a plurality of mutations. In some embodiments, the plurality of mutations comprises one selected from the group consisting of an insertion mutation, a deletion mutation, a point mutation, multiple points mutations, and combinations thereof.

In some embodiments, each target site further comprises a barcode sequence linked to the guide sequence.

In some embodiments, the barcode sequence comprises a nucleotide sequence having a length between about 400 nucleic acids to about 2,000 nucleic acids. In some embodiments, the barcode sequence nucleic acids a nucleotide sequence having a length between about 50 nucleic acids to about 200 nucleic acids.

In some embodiments, each target site in a plurality of target sites within at least one genetic scratchpad comprises the same guide sequence that is recognized by a unique guide molecule.

In some embodiments, each target site in a plurality of target sites within at least one genetic scratchpad comprises a different guide sequence that is recognized by a unique and different guide molecule.

In some embodiments, the plurality of target sites within at least one genetic scratchpad comprises one selected from the group consisting of two or more different guide sequences, three or more different guide sequences, five or more different guide sequences, eight or more different guide sequences, 10 or more different guide sequences, 15 or more different guide sequences, 20 or more different guide sequences, and 30 or more different guide sequences.

In some embodiments, the characterizing step further comprises the steps of: applying a set of probes to cells in the cell population and characterizing a mutation status at the plurality of target sites based on the absence and presence of signals.

In some embodiments, each probe comprises a nucleic acid sequence designed to bind to a target site within the plurality of target site. In some embodiments, each probe is associated with a label that produces a signal upon binding between the probe and its corresponding target site.

In some embodiments, absence of a signal indicates a mutation at the target site and the presence of a signal indicates an intact target site, or vice versa

In some embodiments, the set of probes comprises RNA probes or DNA probes. In some embodiments, probes in the set of probes are associated with multiple labels that produce different signals.

In some embodiments, each probes of the set of probes are designed to bind to a guide sequence within a target site within the plurality of target site.

In some embodiments, each probes of the set of probes are designed to further bind to a barcode sequence linked to the guide sequence within a target site within the plurality of target site.

In one aspect, provided herein is a system for characterizing lineage information or recording molecular events among cells in a cell population. The system comprises a few component, including for example, a housing component, a characterization component and an analytical component.

In some embodiments, the housing component provides housing for one or more cells in a cell population. A plurality of molecular changes is introduced over a time period of multiple cell cycle generations in at least one of one or more genetic scratchpads in one or more cells in a cell population. In some embodiments, the cell population comprises cells that have developed for one or more cell cycle generations. In some embodiments, each genetic scratchpad in the one or more genetic scratchpads comprises a polynucleotide sequence and a plurality of target sites within the polynucleotide sequence. In some embodiments, each of the plurality of molecular changes is associated with a target site among the plurality of target sites.

In some embodiments, the characterization component is configured to characterize the cell population. At one or more time points during the time period, a status of molecular changes at each time for the plurality of target sites in each genetic scratchpad in cells in the cell population is characterized, for example, by fluorescence imaging techniques using probes that recognize mutations with target sites in genetic scratchpads in cells in the cell population. In some embodiments, the molecular changes represent one or more molecular events: they are either the cause or result of one or more molecular events.

In some embodiments, the cells are essentially intact or undisrupted, wherein at least one time point in the one or more time points is two or more cell cycle generations from the beginning of the time period.

In some embodiments, the analytical component is designed to receive data from the characterization component. The analytical component establish lineage connections between cells from different cell cycle generations by comparing mutation statuses of the cells.

Without any limitation, embodiments disclosed herein can be applied to any aspect of the invention, alone or in any combinations.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts an exemplary process.

FIG. 2A depicts an exemplary embodiment of a scratchpad design.

FIG. 2B depicts an exemplary embodiment of a scratchpad design with guide RNA (gRNA) binding sequences.

FIG. 2C depicts an exemplary embodiment of a scratchpad design with guide RNA (gRNA) binding sequences and barcode sequences.

FIG. 2D depicts an exemplary embodiment of a target site within a genetic scratchpad.

FIG. 3A depicts the mechanism for a Clustered Regularly Interspaced Short Palindromic Repeats (CRISPR) system.

FIG. 3B depicts an exemplary expression cassette for gRNA expression.

FIG. 3C depicts an exemplary expression cassette for Cas9 protein expression.

FIG. 4A depicts an exemplary embodiment with multiple gRNAs.

FIG. 4B depicts an exemplary embodiment of a genetic scratchpad with multiple gRNA binding regions.

FIG. 4C depicts an exemplary embodiment, illustrating mutations in multiple cell cycle generations.

FIG. 4D depicts an exemplary embodiment with a single gRNA.

FIG. 4E depicts an exemplary embodiment of a genetic scratchpad with a gRNA binding region coupled with multiple barcode sequences.

FIG. 4F depicts an exemplary embodiment, illustrating mutations in multiple cell cycle generations.

FIG. 5A depicts an exemplary embodiments, illustrating multiple rounds of probe hybridization.

FIG. 5B depicts exemplary schematic images from multiple rounds of probe hybridization.

FIG. 5C depicts exemplary embodiments, illustrating the color code representing a particular target site.

FIG. 6A depicts an exemplary embodiment with multiple gRNAs.

FIG. 6B depicts an exemplary embodiment, illustrating multiple genetic scratchpads each containing one of a few distinct gRNA binding region.

FIG. 6C depicts an exemplary embodiment, illustrating mutations in multiple cell cycle generations.

FIG. 7A depicts an exemplary embodiment of a genetic scratchpad.

FIG. 7B depicts an exemplary linage tree.

FIG. 8A depicts an exemplary embodiment, illustrating deletion mutation in a genetic scratchpad in mammalian cells.

FIG. 8B depicts an exemplary embodiment, illustrating deletion mutation in a genetic scratchpad in yeast cells.

FIG. 9 depicts an exemplary embodiments, showing the effects of mismatched gRNAs.

FIG. 10A depicts an exemplary embodiment, showing FISH image detection of genetic scratchpad in mammalian cells.

FIG. 10B depicts an exemplary embodiment, showing FISH image detection of genetic scratchpad in yeast cells.

FIG. 11A depicts an exemplary embodiment, showing FISH image detection of genetic mutation within genetic scratchpad in mammalian cells.

FIG. 11B depicts an exemplary embodiments, showing FISH image detection of genetic mutation within genetic scratchpad in mammalian cells.

FIG. 12A depicts an exemplary embodiment, showing snapshots of single cells with genetic scratchpads dividing over time.

FIG. 12B depicts an exemplary embodiment, showing FISH image detection of genetic mutation within genetic scratchpad in mammalian cells.

FIG. 12C depicts an exemplary linage tree.

FIG. 13 depicts an exemplary embodiment, illustrating barcoding in cells.

FIG. 14A depicts an exemplary embodiment, illustrating computer-simulated mutations over multiple generations.

FIG. 14B depicts an exemplary embodiment, illustrating a lineage constructed based on the computer-simulated mutation data from FIG. 14A.

### DETAILED DESCRIPTION OF THE INVENTION

Unless otherwise noted, terms are to be understood according to conventional usage by those of ordinary skill in the relevant art.

As used herein, the term “an essentially intact or undisrupted cell” refers to a cell that is completely intact or largely conserved with respect to its macromolecular cellular content. For example, a cell within the meaning of this term can include a cell that is made at least partially permeable such that external buffer and reagents can be introduced into the cell. Such external reagents include but are not limited to probes, labels, labeled probes, and/or combinations thereof.

As used herein, the term “genetic scratchpad” refers to a polynucleotide sequence within a prokaryotic or eukaryotic cell. In some embodiments, the genetic scratchpad can be synthesized in vitro and then put into the cell. In some embodiments, the genetic scratchpad refers to a defined location within the natural genomic sequence of the cell. In some embodiments, the genetic scratchpad can refer to a defined location within the natural genomic sequence of the cell that has been modified. Within the polynucleotide sequence of a genetic scratchpad, there are multiple target sites. In some embodiments, each target site comprises a guide sequence that can be recognized by a unique guide molecule.

As use herein, the term “molecular event” refers to occurrences that happen in a cell and that we can record with our method, like a signaling event, transcription factor activity or even a more complex process such as tumor genesis or kinase transduction pathway. The term “molecular change” or “molecular alteration or mutation” refers to a change that occurs in the scratchpad, like a genetic mutation or genetic modification. The molecular change can be the result or the cause of a molecular event.

As used herein, the term “mutation” or “genetic mutation” refers to any recognizable variation in nucleotide sequence that can be used in accordance with the present invention. For example, a mutation can be a deletion or an insertion of a polynucleotide sequence. In some embodiments, the absence or presence of the polynucleotide sequence can be indicated by using one or more visible indicia; for example, a nucleotide hybridization probe with a fluorescent color label. The length of the polynucleotide deletion or insertion can vary with applications and sensitivities of the probes. For example, the polynucleotide comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, 200 or fewer nucleic acids, 250 or fewer nucleic acids, 300 or fewer nucleic acids, 350 or fewer nucleic acids, 400 or fewer nucleic acids, 450 or fewer nucleic acids, 500 or fewer nucleic acids, 600 or fewer nucleic acids, 700 or fewer nucleic acids, 800 or fewer nucleic acids, 900 or fewer nucleic acids, 1,000 or fewer nucleic acids, 1,500 or fewer nucleotides, 2,000 or fewer nucleic acids, 5,000 or fewer nucleic acids, or 10,000 or fewer nucleic acids. In some embodiments, the polynucleotide insertion or deletion is longer than 10,000 nucleic acids.

As used herein, the term “guide sequence” refers to a sequence within a target site that can be recognized by a molecule or set of molecules that create or trigger molecular changes such as genetic mutations or modifications that lead to certain molecular events such as signal transduction, tumor genesis or metastasis, and etc. Alternatively, molecular events can be the cause of certain molecular changes. This guide molecule may be a guide RNA (gRNA), which recruits a second molecule such as nuclease to the binding site to create mutations. In some embodiments, a guide sequence comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, or 250 or fewer nucleic acids. In some embodiments, the guide sequence comprises 500 or more nucleic acids or even 1,000 nucleic acids when tandem gRNAs are implemented in a target site.

As used herein, the term “barcode” refers to a sequence within a target site that can be used to identify the particular target site. A barcode sequence is also referred to as a target sequence. In some embodiments, a barcode sequence is linked to a corresponding guide sequence. In some embodiments, a barcode sequence comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, 250 or fewer nucleic acids, 500 or fewer nucleic acids, 1,000 or fewer nucleic acids, 1,500 or fewer nucleic acids, 2,000 or fewer nucleic acids, or 5,000 or fewer nucleic acids. In some embodiments, a barcode sequence comprises more than 5,000 nucleic acids.

As used herein, the term “probe” refers to any composition that can be specifically associated with a target nucleotide within a cell. A probe can be a small molecular or a large molecule. Exemplary probes include but are not limited to nucleic acids such as oligos. In some embodiments, a probe is associated with a visible label such as a fluorescence label to indicate the presence of a certain nucleotide sequence. In some embodiments, the probe can be a DNA probe or an RNA probe. In some embodiments, a probe sequence comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, 250 or fewer nucleic acids, or 500 or fewer nucleic acid. In some embodiments, a probe comprises more than 500 nucleic acids.

As used herein, the term “label” refers to any composition that can be used to generate the signals that constitute an indicium. The signals generated by a label can be of any form that can be resolved subsequently to constitute the indicium. Preferably, the signal is a light within the visible range. However, it will be understood by one of skill in the art that equipment and devices are available for recording and monitoring light of any wavelength. The label can also constitute any moiety, such as a hapten, that can be recognized by an antibody. This secondary antibody can be conjugated to a fluorescent molecule or an enzyme that can produce signals that constitute an indicium.

Disclosed herein are methods and systems for capturing molecular events within cells to extrapolate lineage information between cells from different generations. An exemplary system includes one or more of the following components: one or more genetic scratchpad(s) where molecular changes such as genetic mutations or modification will occur; a writing component for creating the genetic mutations within the genetic scratchpad; a characterization component for capturing the mutation status of a genetic scratchpad by identifying the presence and absence of such genetic mutations; and an analysis component for reading out mutations that have been created in the scratchpads.

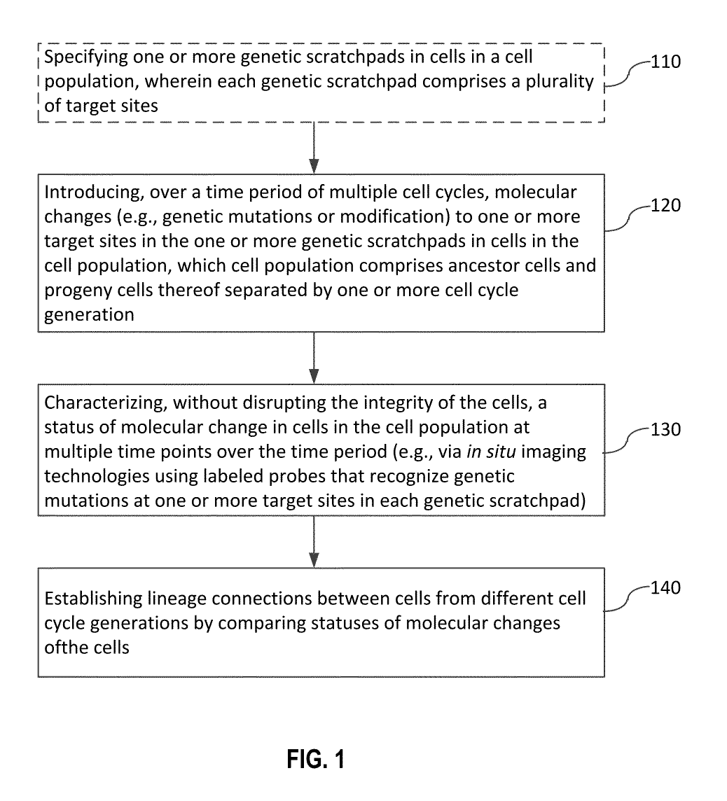

FIG. 1 outlines an exemplary process disclosed herein.

At step 110, one or more genetic scratchpads are specified with a cell. As noted above, molecular changes as disclosed herein (e.g., genetic mutations or modification) take place within the genetic scratchpads. More precisely, a genetic scratch comprises one or more target sites and the molecular changes take place at the target sites. One of skill in the art will understand that similar molecular changes also occur elsewhere inside the cells. However, those events are not within the scope of subsequent analysis. In addition, after the molecular changes have taken place, subsequent analysis (such as visualization of the presence and absence of genetic mutations) will also be focused on the genetic scratchpad, for example at the target sites. As disclosed herein, the terms “genetic scratchpad,” “scratchpad” and variations thereof are used interchangeably.

As disclosed herein, a genetic scratchpad comprises nucleotide sequences that are synthesized in vitro. Alternatively, a genetic scratchpad comprises a natural region of the genomic sequence of the cell. Still alternatively, a genetic scratchpad comprises a hybrid of synthetic and natural sequences. Still alternatively, a genetic scratchpad comprises natural nucleotide sequence that has been modified at one or more locations.

At step 120, molecular changes such as genetic mutations are introduced into one or more genetic scratchpads over a time period that spans multiple cell cycle generations. Such molecular changes can be genetic mutations such as insertions or deletions of nucleotide sequences at one or more of the target sites within a genetic scratchpad. Alternatively, the molecular changes can be genetic modifications. For example, a DNA segment can be methylated to alternative its functionality or possibility of be transcribed. In particular, a methyl-transferase can be fused to cas9 and target specific sites to bring about changes in a target site in one or more genetic scratchpads.

At any given cell cycle, the same molecular changes can be introduced into multiple genetic scratchpads or multiple target sites within the same scratchpad. In some embodiments, no molecular changes take place in any genetic scratchpad during a particular cell cycle.

At step 130, the genetic status of the genetic scratchpads (e.g., the status of target sites within the scratchpads) within cells from step 120 is characterized. Characterization of genetic status includes identifying the presence and absence of genetic mutations at target sites within one or more scratchpads.

In some embodiments, labeled probes designed to bind specific sequences in the target sites are used. For example, an intact target site (e.g., no molecular change has taken place at the site) will allow proper binding between the labelled probes and the target site. Upon binding, the label can be induced to emit signals such as fluorescent light. In contrast, if a target site is disrupted by a molecular change, for example, due to deletion or insert of nucleotide sequences, a probe specifically targeting the site will no longer be able to bind. Consequently, there will be no label attached to the target site and no subsequent fluorescent signals. In exemplary embodiments, the presence of fluorescent signal at a target site suggests that no molecular changes have occurred while absence of such a signal at a target site suggests that one or more molecular changes have occurred to disrupt the sequence at the target site. In alternate embodiments, the induced mutation could result in the emergence of a new, detectable fluorescence signal. For example, in the absence of a mutation, fluorescent probes might not bind the target site. After a particular mutation, such as an insertion mutation, probes will be able to bind the site and produce a detectable signal.

Over multiple cell cycles, a cell (e.g., an ancestor cell) at the beginning of the time period has divided into multiple progeny cells. As such, at a given time point, there are progeny cells present that carry information about their past and ancestry. As disclosed herein, characterization of genetic status is carried out for cells in the cell population at a defined time point. Genetic status characterization of cells within the population allows construction of their lineage relationships as well as a record of any other historical events being tracked. The characterization time point is selected to provide information across the time window of interest, which ideally spans multiple cell cycle generations to allow reconstruction of a comprehensive history.

Alternatively, characterization can also be carried out at multiple, distinct time points. The time points can be chosen as desired to focus on changes across cell generations of interest. In some embodiments, this can be helpful in order to effectively sample changes across long processes and/or focus on multiple subsets of events within these processes: for example, for extracting lineage information and cellular histories during stereotypic, developmental processes, where defined cell types emerge at distinct times.

In some embodiments, presence and absence of fluorescent signals are determined by comparing images of both ancestor and progeny cells.

Here, the genetic status of a given cell is assessed while the structural and functional integrity within the cell is maintained. Additionally minimal perturbations are made to the spatial proximity of the cells within the population.

At step 140, the genetic status data captured at step 130 is subject to further analysis. In particular, the mutation status of an ancestor cell and its progeny cells at different cell cycle generations are identified and compared to extrapolate lineage and phylogenetic information and/or cellular event history.

In one aspect, the method and system disclosed herein are capable of capturing or recording multiple molecular changes over time; it is not limited to registering a single change.

To this end, in some embodiments, multiple “scratchpads” are specified in the cell genome. A genetic scratchpad can be any polynucleotide sequence whose sequence information is at least partially known. A scratchpad can be “written on” and serves as a unique recording or capturing site.

Scratchpads can be synthetic and composed of a variety of elements including repetitive segments, homology regions flanking a central core comprising the repetitive segments and one or more promoter sequences, and enzymatic recognition sequences. Scratchpad units may be a range of lengths and include various upstream promoters or other elements and different downstream sequences. They can be introduced into the genome as separate units or as part of a larger integrated cassette, like an artificial chromosome. Alternatively, scratchpads can also utilize the endogenous genomic DNA and not require synthetic additions.

In some embodiments, a genetic scratchpad comprises nucleotide sequences that are synthesized in vitro and then introduced into cells by methods such as transfection.

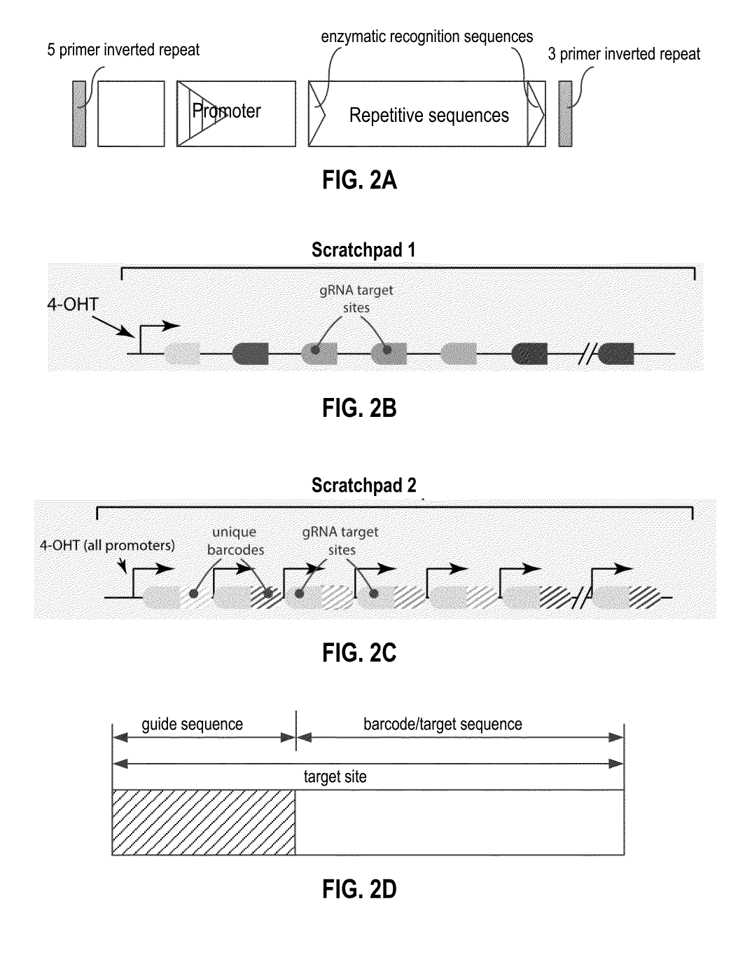

FIG. 2A depicts an exemplary embodiment, illustrating the basic scratchpad configuration, from left to right, which includes a 5 prime inverted repeat for integration (thin rectangle), an insulated promoter region (rectangular box with an arrow), a repetitive region flanked by enzymatic recognition sequences (thin arrowheads), and 3 prime inverted repeat (thin rectangle).

In some embodiments, an implementation of this strategy involves a scratchpad with a repetitive sequence at its core that can be deleted (FIG. 2A); for example, by enzyme that can recognize the recognition sequences that flank the repetitive sequences. In some embodiments, the scratchpad has multiple target sites and the repetitive sequences are inserted at different target sites in the scratchpad. In some embodiments, such repetitive sequences are inserted into multiple scratchpads.

In some embodiments, an implementation of this strategy involves a scratchpad with a repetitive sequence at its core that can be deleted (FIG. 2A). In such embodiments, a genetic scratchpad comprises one or more target sites with such a repetitive sequence. In some embodiments, these target sites comprise different number of copies of such a repetitive sequences. For example, scratchpad A has 5 target sites. Target site 1 has 3 copies of the repetitive sequences while target site 2 can have 5 or more copies of the same repetitive sequences and etc. Because the repetitive sequences are between enzyme cleavage sites, by altering the number of repetitive sequences, different target sites can be identified by using methods that can assess the length of the resulting genetic scratchpad. An exemplary method includes single cell based polymerase chain reaction (PCR) analysis.

In some embodiments, though the core of the scratchpad is the same in each case, the sites can actually be differentiated because they are flanked by distinct genomic regions. The genomic context of each scratchpad can be identified individually by PCR and/or next generation sequencing methods, providing a unique target sequence or “barcode” for each scratchpad. For example, one characterized line has at least 10 scratchpads spread across unique genomic regions on 7 chromosomes. Unique target sequence or barcodes can also be created by other means, including constructing scratchpads with different unique synthetic sequences.

In some embodiments, multiple copies of this scratchpad can be introduced throughout the genome by transposase mediated recognition of inverted repeats (FIG. 2A), or other means, creating a large number of unique target sites. Molecular changes at these target sites will be captured or recorded.

In some embodiments, the scratchpad can contain other features, such as a promoter that allows transcription of this scratchpad and helps with readout (a feature described further below).

In alterative embodiments, a genetic scratchpad is located in defined regions within the natural genome of a cell. Because the sequence information of the genome of many organisms, including humans, is known, a genetic scratchpad can be defined based on the sequence information of selected genetic regions of interest in a genome. For example, sequences near or at genetic regions of interest (e.g., a target site) can be designated as a guide sequence to recruit one or more secondary molecules (e.g., a guide RNA known as a gRNA and a nuclease that is recruited by the gRNA), which facilitate the occurrence of certain molecular changes at the genetic regions of interest. In some embodiments, a nick or a double stranded break is created by the one or more secondary molecules resulting in disruption of the genetic region of interest, which can then be detected by the characterization component.

In still alternative embodiments, synthetic guide sequences can be inserted into selected regions within the natural genome of a cell. In some embodiments, such guide sequences are located at or near regions of interest such as target sites. As disclosed herein above, the guide sequences can recruit one or more secondary molecules (e.g., a guide RNA known as a gRNA and a nuclease that is recruited by the gRNA), which facilitate the occurrence of certain molecular changes at the genetic region of interest.

As disclosed herein, a cell can have one or more genetic scratchpads. In some embodiments, a cell has two or more genetic scratchpads, such as between three and five genetic scratchpads. In some embodiments, a cell has five or more genetic scratchpads, such as between five and nine genetic scratchpads. In some embodiments, a cell has 10 or more genetic scratchpads, such as between 10 and 15 genetic scratchpads. In some embodiments, a cell has 15 or more genetic scratchpads, such as between 15 and 19 genetic scratchpads. In some embodiments, a cell has 20 or more genetic scratchpads, 25 or more genetic scratchpads, 30 or more genetic scratchpads, 40 or more genetic scratchpads, 50 or more genetic scratchpads, 60 or more genetic scratchpads, 70 or more genetic scratchpads, 80 or more genetic scratchpads, 90 or more genetic scratchpads, 100 or more genetic scratchpads, 120 or more genetic scratchpads, 150 or more genetic scratchpads, 180 or more genetic scratchpads, 200 or more genetic scratchpads, or 500 or more genetic scratchpads.

In some embodiments, the number of genetic scratchpads in a particular genomic is determined by the complexity of the lineage information. For example, the number of genetic scratchpads required for assessing the lineage information cross 10 possible regions of interest will be larger than that required for assessing the lineage information cross 3 or 5 possible regions of interest.

In some embodiments, the entire sequence information of the genetic scratchpad is known. In some embodiments, only a part of the sequence information of the genetic scratchpad is known.

Also as disclosed, a genetic scratchpad comprises a polynucleotide sequence of any length. In some embodiments, the polynucleotide comprises 100 nucleotides or longer; 200 nucleotides or longer; 300 nucleotides or longer; 400 nucleotides or longer; 500 nucleotides or longer; 700 nucleotides or longer; 1,000 nucleotides or longer; 1,500 nucleotides or longer; 2,000 nucleotides or longer; 2,500 nucleotides or longer; 3,000 nucleotides or longer; 4,000 nucleotides or longer; 5,000 nucleotides or longer; 6,000 nucleotides or longer; 7,000 nucleotides or longer; 8,000 nucleotides or longer; 10,000 nucleotides or longer; 12,000 nucleotides or longer; 15,000 nucleotides or longer; 20,000 nucleotides or longer; 50,000 nucleotides or longer; or 100,000 nucleotides or longer.

Preliminary modeling suggests that, in order to allow proper tracking of lineage information, an ideal system would provide at least two mutations per generation per scratchpad. To track about 10 generations, about 100 target sites should be sufficient.

A genetic scratchpad comprises multiple target sites, as depicted in the exemplary genetic scratchpads in FIGS. 2B and 2C. In some embodiments, each target site comprises a binding site that is recognized by a guide molecule such as a guide RNA (gRNA). In some embodiments, each target site comprises a target sequence or barcode associated with a guide molecule binding site.

FIG. 2D illustrates an exemplary target site, for example, those corresponding to those depicted in FIG. 2C. In such embodiments, the target site comprises a guide sequence with a segment that is recognized by a gRNA. In some embodiments, the gRNA has a complementary sequence that allows the gRNA to bind to the guide sequence. In some embodiments, the sequence in the gRNA can be adjusted to modify the binding interactions between the gRNA and the guide sequence within a target site. Such adjustment is used to modulate the frequency at which the gRNA binds to the guide sequence and thereby modulating the frequency at which any molecular events that may occur upon binding between the gRNA and the guide sequence.

In some embodiments, when a gRNA binds to its corresponding guide sequence, it recruits one or more secondary molecules, which then trigger one or more molecular changes. For example, an enzyme such as Cas9 nuclease can be recruited to the gRNA binding site. The nuclease then creates nicks or double-stranded break at the binding site, thereby destroying the structural integrity of a target site.

In some embodiments, all or at least a part of the guide sequence is also recognized by a molecule that is used to characterize the integrity of a target site. For example, such a molecule can be a hybridization probe for fluorescence imaging analysis.

In some embodiments, a target site further comprises a barcode or target sequence. All or at least a part of the barcode or target sequence is also recognized by a molecule that is used to characterize the integrity of a target site. For example, such a molecule can be a hybridization probe for fluorescence imaging analysis.

In some embodiments, the length of the guide sequence is typically at least 20 nucleotides. However, guide sequences can be shorter or longer to modify their associated efficiency in recruiting secondary molecules. Additionally, to target multiple sequences, with a signal guide RNA molecule, guide sequences can be arranged in tandem with intervening spacer regions.

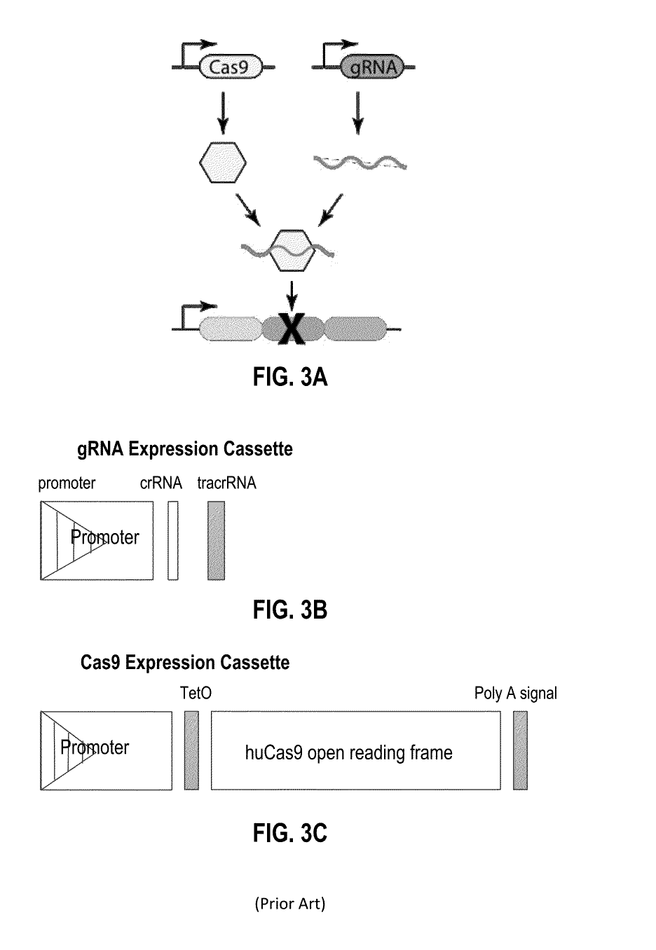

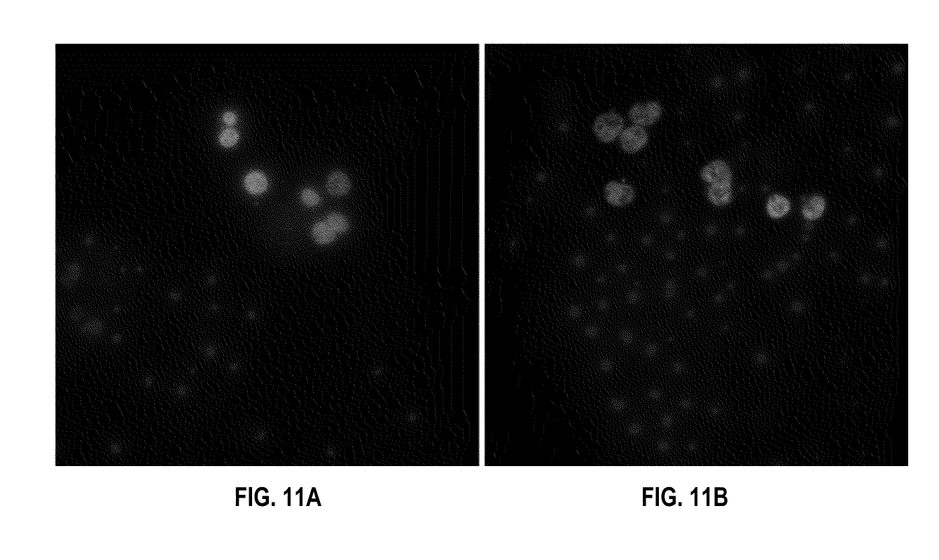

In some embodiments where multiple scratchpads are present in a genome, each scratchpad can be independently written (e.g., via enzymatic cleavage of repetitive sequences) or using a genomic editing tool such as the Clustered Regularly Interspaced Short Palindromic Repeats (CRISPR) system (e.g., through a guide RNA and the Cas9 nuclease) (FIGS. 3A-3C). Presence of Cas9 and a specific guide RNA (gRNA) in the system leads to deletion of the scratchpad core, a change readily detected in bulk (FIG. 3) and in situ (FIG. 11).

In one aspect, provided herein is a writing component that is capable of creating the molecular changes to be captured or recorded.

In order to capture or record the molecular changes, a writing component should trigger or create molecular changes only in defined regions, for example, within a target site. This way, changes brought about by the molecular changes can be assessed in subsequent characterization analysis. To this end, a writing component comprises a guide molecule. The main function of the guide molecule is to recognize a desired target site. In some embodiments, the guide molecule is an RNA molecule that associates itself to the desired target site via complementary sequence recognition. In some embodiments, other molecules may facilitate the recognition and association between the guide molecule and the desired target site.

In addition, the writing component comprises one or more secondary molecules that are capable of triggering or creating one or more molecular changes at the desired target site. In some embodiments, one or more secondary molecules are recruited by the guide molecule to the target site. In some embodiments, the guide molecule binds to a guide sequence first to form a complex, which is then recognized by one or more secondary molecules. In some embodiments, the guide molecule and one or more secondary molecules bind first before the complex recognizes and binds to the guide sequence at the target site.

In some embodiments, the Clustered Regularly Interspaced Short Palindromic Repeats (CRISPR) system, one of the most commonly used RNA-Guided Endonuclease technologies for genome engineering, can be used as a writing component. Exemplary embodiments of the CRISPR system are depicted in FIGS. 3A through 3C.

In a CRISPR system, the guide molecule is a gRNA (e.g., FIG. 3A). When the gRNA binds to a guide sequence in the target site, it recruits secondary molecules (e.g., Cas9 nuclease) to trigger subsequent molecular changes: nicks or break in nucleotide sequences, which leads to various genetic mutations. Such genetic mutations include but are not limited to insertion mutation, deletion mutation, point mutations, multiple point mutations, any combination of such mutations, or any other changes at the nucleic acid level that can affect the binding of guide molecules such as gRNAs. Insertion and deletion mutations (also referred to as indel mutations) often lead to frame shift mutations leading to major disruptions in one or more genes, as illustrated in FIG. 3A. As such, probes designed to recognize the original target site will no longer be able to bind to the disrupted region. Alternatively, molecular changes include genetic modification. For example, a methyl-transferase can be fused to cas9 and target specific sites to alter the subsequent activity of a target site in one or more genetic scratchpads. Methylation on the DNA can be detected by bi-sulfite conversion, which turns unmethylated Cs to Us.

A typical CRISPR system comprises two independent cassettes for expressing its two distinct components: (1) a guide RNA and (2) an endonuclease such as the CRISPR associated (Cas) nuclease, Cas9.

The guide RNA is a combination of the endogenous bacterial crRNA and tracrRNA into a single chimeric guide RNA (gRNA) transcript. The gRNA combines the targeting specificity of the crRNA with the scaffolding properties of the tracrRNA into a single transcript. An exemplary gRNA expression cassette (e.g., FIG. 3B) depicts an RNA polymerase III or polymerase II specific promoter (box with an arrowhead), which drives the expression of a chimeric crRNA (middle rectangle) and tracrRNA (far right, shaded rectangle).

An exemplary Cas9 expression cassette is found in FIG. 3C, which shows an RNA polymerase II promoter (rectangle with an arrowhead), an array of two binding sites for a repressor protein (TetR) and a “humanized” huCas9 open reading frame followed by poly A signal from the bovine growth hormone gene (dark, shaded rectangle). When the gRNA and the Cas9 nuclease are expressed in the cell, the genomic target sequence can be modified or permanently disrupted.

The gRNA/Cas9 complex is recruited to the target sequence by the base-pairing between the gRNA sequence and the complement to the target sequence in the genomic DNA. In some embodiments, to ensure successful binding of Cas9, the genomic target sequence also contains the correct protospacer adjacent motif (PAM) sequence immediately following the target sequence. The binding of the gRNA/Cas9 complex localizes the Cas9 to the genomic target sequence so that the wild-type Cas9 can cut both strands of DNA causing a double strand break (DSB). Cas9 cuts 3-4 nucleotides upstream of the PAM sequence.

Recent publication (13, 14) and preliminary experiments suggest that Cas9 can be a suitable component for “writing” random mutations into an engineered scratchpad region in the genome, where the scratchpad comprises many individually addressable target sites for the gRNA-Cas9 complex (FIGS. 2B and 2C). Aspects of the Cas9 system enable tuning of the rate of mutagenesis and scaling of the size of the target region.

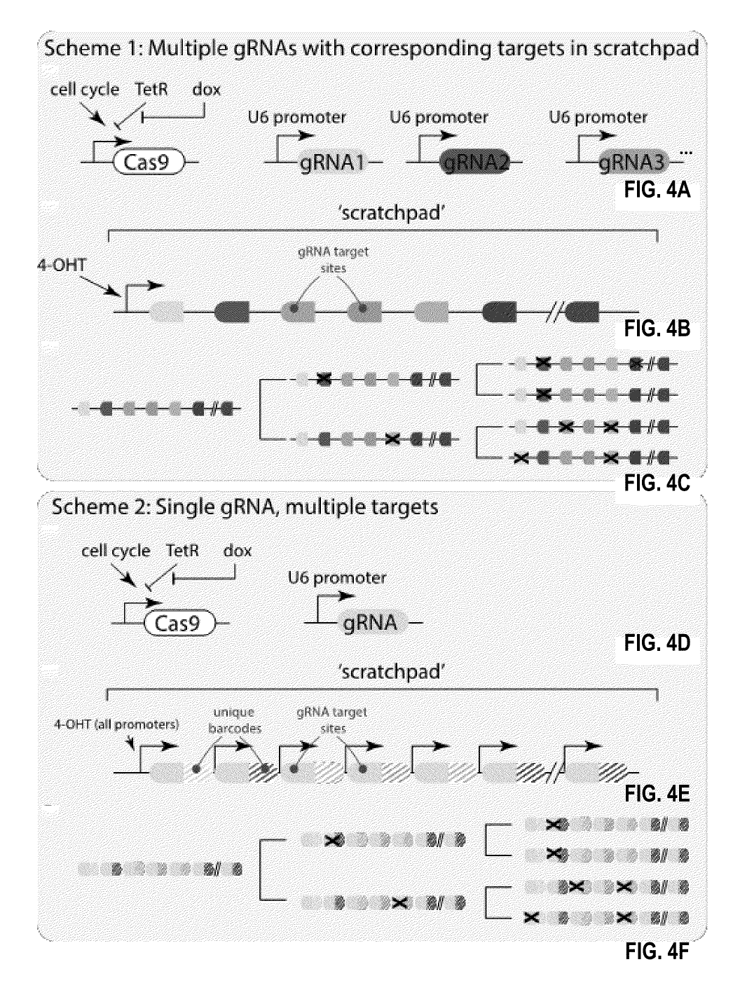

FIGS. 4A through 4F illustrate two exemplary schemes for creating genetic mutations into genetic scratchpads. In each one, a set of expression constructs (FIGS. 4A and 4D), a corresponding scratchpad (FIGS. 4B and 4E) and a schematic 3-generation lineage tree (FIGS. 4C and 4F) are shown. X's indicate mutations.

In Scheme 1, the CRISPR system includes one Cas9 protein but multiple gRNAs (e.g., FIG. 4A). In some embodiments, the gRNAs are all under the control of a U6 promoter. Each gRNA binds to a unique target site in a genetic scratchpad and subsequently recruits the Cas9 nuclease to create a mutation at the target site (e.g., FIG. 4B). The site of the mutations may depend on the binding efficiency of the particular gRNA or the cutting efficiency of the Cas9 nuclease at the site.

In some embodiments, multiple mutations accumulate over multiple cell cycle generations. For example, as illustrated in FIG. 4C, the genetic scratchpad of FIG. 4B leads to two possible mutations in its first generation offspring: one comprising a mutation at target site No. 2 and the other comprising a mutation at target site No. 5. The mutations are preserved in the offspring of these two first generation offspring.

In some embodiments, additional mutations are created in addition to those carried over from the parent generation. In some embodiments, no additional mutations are created in one or more generations. For example, as depicted in FIG. 4C, in the next generation, no additional mutation is introduced into the scratchpad containing the mutation at target site No. 2. However, the scratchpad carrying the mutation at target site No. 5 leads to two offspring with double mutations: one with mutations at target site No. 3 and site No. 5 and the other at target site No. 1 and No. 5.

In some embodiments, it is also possible for multiple mutations to occur in subsequent generations, such as two or more mutations, three or more mutations, or even five or more mutations. In order to keep the number of mutations under a reasonable limit and better assess lineage information between different generations, various methods (e.g., by applying mismatching sequences in a gRNA to adjust the rate at which it binds to a guide sequence) are applied to adjust the occurrence rate of mutations.

In Scheme 2, only a single gRNA is used against multiple target sites (e.g., FIG. 4D). Here, instead of having unique gRNAs bind to different target site, each target site includes a unique barcode or target sequence to which unique probes can bind to reveal the presence of a particular target site (e.g., FIG. 4E). The detailed recognition mechanism will be described in the following section.

Similar to the setup of Scheme 1, binding of the gRNA to a target site also ultimately leads to mutations after a Cas9 nuclease is recruited. Also similarly, such mutations can be preserved in future generations. Further, additional mutations can occur at different target sites in future generations of cells.

As illustrated, lineage trees can be inferred from determination of the patterns of mutations (e.g., FIGS. 4C and 4F).

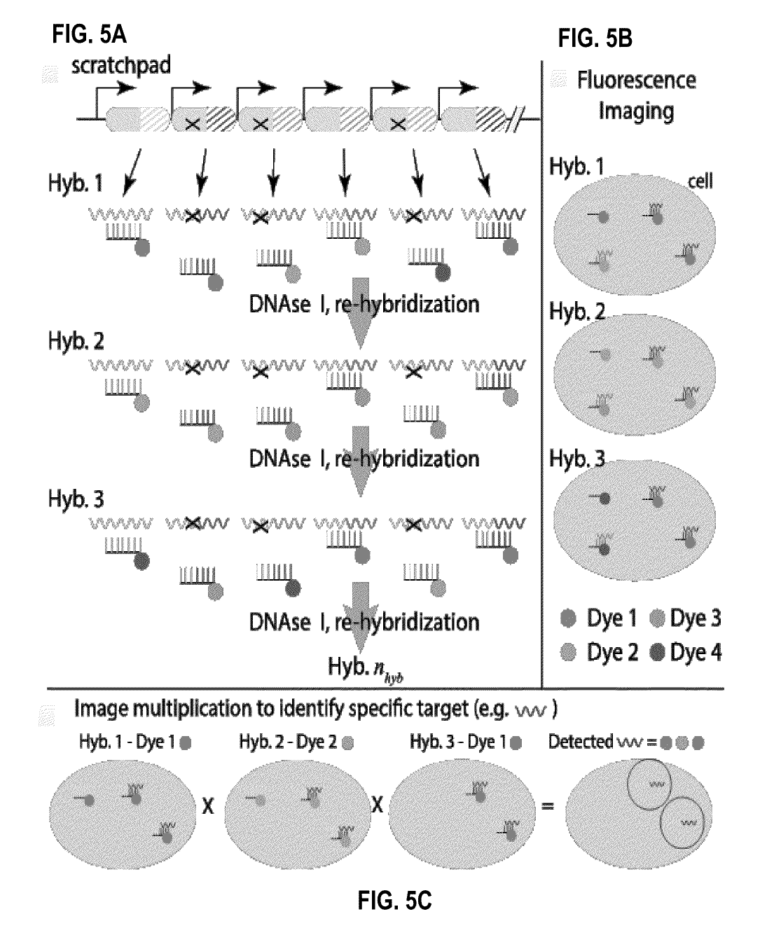

Scheme 1 is optimized for single-cell DNA sequencing detection of mutations, while Scheme 2 is optimized for detection by multiplexed FISH (e.g., FIG. 5). In both schemes, the scratchpads can be transcribed from a promoter. The promoter can be either inducible or constitutive. Expression enables mutations to be read out by hybridization to RNA (FIG. 5).

In one aspect, provided herein are methods and systems for characterizing the location of mutations in one or more genetic scratchpads.

In some embodiments, single-cell sequencing techniques can be used to reveal the mutations in the target sites in one or more scratchpads before standard computational methods are applied to determine lineage relationships.

In some embodiments, to readout the mutations made on the scratchpad in situ, a recently developed method is adapted to identify mutations in single cells within complex tissues while preserving spatial information. In some embodiments, the expression of the recording region into RNA is induced from an upstream inducible promoter (e.g., FIGS. 4A and 4D). This has two benefits. First, it allows the application of single molecule fluorescent in situ hybridization (smFISH or FISH) (18), which is already optimized for RNA detection. In addition, transcription amplifies the signal, as multiple copies of each mRNA are expressed from the scratchpad region, which enhances detection efficiency and accuracy.

To uniquely distinguish the different target sites on the scratchpad, unique barcode sequences are engineered at each target site (FIG. 4E). FISH probes recognizing such unique sequence are designed to span the junction across the target site and the barcoded region, and are thus sensitive to mutations in or near the target. In some embodiments, these mutations are large insertions or deletions, which are readily detected by FISH probe hybridization.

In some embodiments, it is possible to detect indels or minor mutations such as single point mutations and multiple point mutations. Recent work has shown that single nucleotide polymorphisms (SNPs) on individual transcripts can be efficiently detected by 25 mer FISH probes (8).

As disclosed herein, indel mutations are suitable molecular changes for a couple of reasons. First, indels are easier to detect than SNPs, since frameshifts are more disruptive to hybridization than mutations. Second, as the RNA is overexpressed from the reading template region, a large number of transcript copies can be analyzed in each cell, boosting the detectable signal.

In some embodiments, probes used to recognize and bind to an mRNA transcript or a DNA sequence are oligonucleotides, or oligos. In some embodiments, the oligo probes are 10-mer or shorter. In some embodiments, the oligo probes are 15-mer or shorter. In some embodiments, the oligos are 20-mer or shorter; 25-mer or shorter; 30-mer or shorter; 40-mer or shorter; 50-mer or shorter; 70-mer or shorter; 100-mer or shorter; 150-mer or shorter; 200-mer or shorter; 250-mer or shorter; 300-mer or shorter; 500-mer or shorter; or 1,000-mer or shorter.

In some embodiments, the oligo probes are designed by using complementary sequences to randomly selected sequences or segment of sequences in a target sequence (e.g., an mRNA or DNA sequence).

In some embodiments, the oligo probes are designed by deliberately selecting sequences or segments of sequences that bind to a target site (e.g., an mRNA or DNA sequence) with known or predicted binding affinity. This is called “intelligent probe design,” where structure, sequence and biochemical data are all considered to create probes that will likely have better binding properties to a target site. In particular, the preferred regions to be used as target sites in a genome are either identified experimentally or predicted by algorithms based on experimental data or computation data. For example, computed binding energy and/or theoretical melting temperature can be used as selection criteria in intelligent probe design.

Tools are available for automated designs of probes that will have either actual or predicted optimal binding properties to the target site. For example, the Designer program is routinely used for designing probes that bind to a particular target RNA sequence as part of the established single molecule RNA Fluorescent in-situ hybridization technology (FISH), which was developed at the University of Medicine and Dentistry of New Jersey (UMDNJ) a Single Molecule Fluorescent in-situ hybridization technology based on detection of RNA (singlemoleculefish<dot>com/designer<dot>html). For the Designer program, the open reading frame (ORF) of the gene of interest is typically used as input. This approach is used to exclude the more repetitive regions and low complexity sequence contained in Un-translated Regions (UTRs). Probes are designed to minimize deviations from the specified target GC percentage. The program will output the maximum number of probes possible up to the number specified. Sequence input is stripped of all non-sequence characters. A user can specify parameters such as the number of probes, target GC content, length of oligonucleotide and spacing length. Most success has been achieved with target GC contents of 45%. Typically, oligos are designed as 20 nucleotides in length and are spaced a minimum of two nucleotides apart.

One of skill in the art would also understand that length or size of probes will vary, depending on the target sites, genetic scratchpad and purposes of the analysis.

Additional description on single molecule FISH can be found in, for example, Raj A., et al., 2008, “Imaging individual mRNA molecules using multiple singly labeled probes,” Nature Methods 5(10): 877-879; Femino A., et al., 1998, “Visualization of single RNA transcripts in situ,” Science 280: 585-590; Vargas D., et al., 2005, “Mechanism of mRNA transport in the nucleus,” Proc. Natl. Acad. Sci. of USA 102: 17008-17013; Raj A., et al., 2006, “Stochastic mRNA synthesis in mammalian cells,” PLoS Biology 4(10):e309; Maamar H., et al., 2007, “Noise in gene expression determines cell fate in B. subtilis,” Science, 317: 526-529; and Raj A., et al., 2010 “Variability in gene expression underlies incomplete penetrance,” Nature 463:913; each of which is hereby incorporated by reference herein in its entirety.

Any suitable labels can be associated with the specific probes to allow them to emit signals that will be used in subsequence imaging analysis. In some embodiments, the same type of labels can be attached to different probes for different target sites.

One of skill in the art would understand that choices for a label are determined based on a variety of factors, including, for example, size, types of signals generated, manners attached to or incorporated into a probe, properties of the target sites including their locations within the cell, properties of the cells, types of interactions being analyzed, and etc.

In some embodiments, all the target sites on the scratchpad are scanned to determine the target sites that are mutated in each cell. In some embodiments, a method to multiplex mRNA detection in single cells in situ is applied. In this approach, the mRNAs in cells are barcoded by sequential rounds of hybridization, imaging, and probe stripping (FIGS. 5A through 5C). As the transcripts are fixed in cells, the fluorescent spots corresponding to single mRNAs remain in place during multiple rounds of hybridization, and can be aligned to read out a color sequence at each point in the cell. This temporal barcode is designed to uniquely identify an mRNA species in a multiplexed experiment. During each round of hybridization, each transcript is targeted by FISH probes labeled with one dye. The sample is imaged and treated to remove the FISH probes. Then the mRNA is hybridized in a subsequent round with the same FISH probes labeled with a different dye. The number of barcodes available with this approach scales as F^{N}, where F is the number of fluorophores and N is the number of hybridization rounds. For example, with 4 dyes, 8 rounds of hybridization can cover the entire transcriptome (4⁸=65,536).

Using FISH and fluorescent microscopy to analyze mutation events has the significant advantage compared to DNA-seq that single cells do not need to be extracted from tissues. Spatial context is preserved. For example, it is possible with this approach to visualize individual cells within a brain slice to determine the mutation set in each of those cells. This not only preserves the spatial information, but is less labor and cost intensive to perform. With conventional fluorescent microscopy, a 1 mm×1 mm×1 mm region can be scanned in approximately 5 minutes. The entire mouse brain can be imaged in 100 hours. With an automated microscope, 4 rounds of hybridization can be performed in 2-3 weeks. The overall cost of the microscope time and reagents will be approximately $10-50 k per brain. In comparison, single cell DNA sequencing costs approximately $10 per cell at the present, and dissecting out more than 1000 cells would be prohibitively labor intensive and cost prohibitive. Lastly, it is possible to apply this approach to CLARITY (9) cleared brains to obtain lineage information directly from intact brains.

FIGS. 5A through 5C depict an exemplary process for detecting mutations in a genetic scratchpad by RNA hybridization FISH. FISH probes used here include sequence that binds to all or a part of guide sequence and all or a part of the barcode or target sequence adjacent or near the guide sequence. Fluorescent signals are only emitted when the FISH probes bind to un-mutated sequences. Disruption of either sequence will lead to loss of signal.

As disclosed previous, disruption by Cas9 results in mutations in the guide sequence (e.g., insertion, deletion or point mutations). Such mutations, in particular, the insertion and deletion mutations prevent a FISH probe from binding to both the guide sequence and/or barcode sequence.

Here, scratchpads are expressed as mRNAs to enable detection of mutations using FISH probes in individual cells. Using sequential rounds of hybridization (Hybs. 1, 2, 3, . . . ) multiple target sites can be probed simultaneously in single cells. In each round of hybridization, a mutation is targeted by a FISH probe with the same sequence but a different dye (e.g., FIG. 5A). Thus, each mutation can be addressed by a particular dye sequence.

For example, the genetic scratchpad here contains 3 mutations, at target sites No. 2, No. 3 and No. 5. In three rounds of hybridization, probes recognizing different target sites are as follows.

| | | Probe Color | Probe Color | Probe Color |
|---|---|---|---|---|
| | Mutation? | (Round 1) | (Round 2) | (Round 3) |
| Target site No. 1 | No | Blue | Green | Red |
| Target site No. 2 | Yes | Blue | Green | Orange |
| Target site No. 3 | Yes | Green | Orange | Red |
| Target site No. 4 | No | Green | Orange | Blue |
| Target site No. 5 | Yes | Red | Orange | Green |
| Target site No. 6 | No | Blue | Green | Blue |

After the mutations, only intact target sites are able to produce fluorescent signals. Sequential hybridizations determine which transcripts are both present and do not contain mutations.

At each hybridization step, cells are imaged in all channels. Color dots in cells correspond to probes hybridizing to indicated transcripts (FIG. 5B). Each round of hybridization results in a snapshot of the cell containing multiple fluorescent signals. Here, it is possible to detect the signal from the same target site multiple times, because multiple copies of mRNA can be synthesized.

Because the characterization is done in situ without disrupting the structural integrity of the cells, it is possible to observe multiple color sequences for the same target site after each round of hybridization. The order by which the color signals appear forms a unique code for identifying the particular target site.

By multiplying or, more generally, cross-correlating images in different rounds of hybridization, one can specifically detect the color sequence of any desired transcript. For example, here the intact target site No. 6 is uniquely detected by combining the blue Hyb 1 image with the green Hyb 2 image and the blue Hyb 3 image (FIG. 5C).

As listed in the table above, by alternating the colors of different probes and applying multiple round of hybridization, each target site corresponds to a particular color sequence code. Here, intact site No. 1 will produce blue, green, and red signals in the order specified. Intact site No. 4 will produce red, orange, and green signals in the order specified. Intact site No. 6 will produce blue, green, and blue signals in the order specified.

One of skill in the art would understand that, when more target sites are involved, more rounds of hybridization will be performed to establish color code sequences that can sufficiently and uniquely identify any intact target site

In some embodiments, other in situ readout methods can also be applied to characterize the mutation status of target sites with one or more genetic scratchpads. Beyond RNA FISH, it is possible to use DNA FISH for in situ readout of recorded events. Expression changes to fluorescence reporters could also be used (in both live and fixed cells), though limits on the number of distinct fluorophore colors could cap the number of recordable events. Other readout methods could also provide in situ-like information, such as single-cell sequencing or PCR when implemented to preserve spatial information. Further, multiple techniques (including single-cell sequencing and PCR) could be readily applied to verify population averages.

Methods and systems described herein enable the reconstruction of lineage trees based on the historical record of induced mutations recorded in scratchpads. More importantly, the recorded information can include data on specific molecular events that occurred in each branch of the tree over time. Exemplary events include but are not limited to activation of master transcription factors or signaling pathways.

To achieve event recording, provided herein are strategies for simultaneously recording lineage information and molecular events.

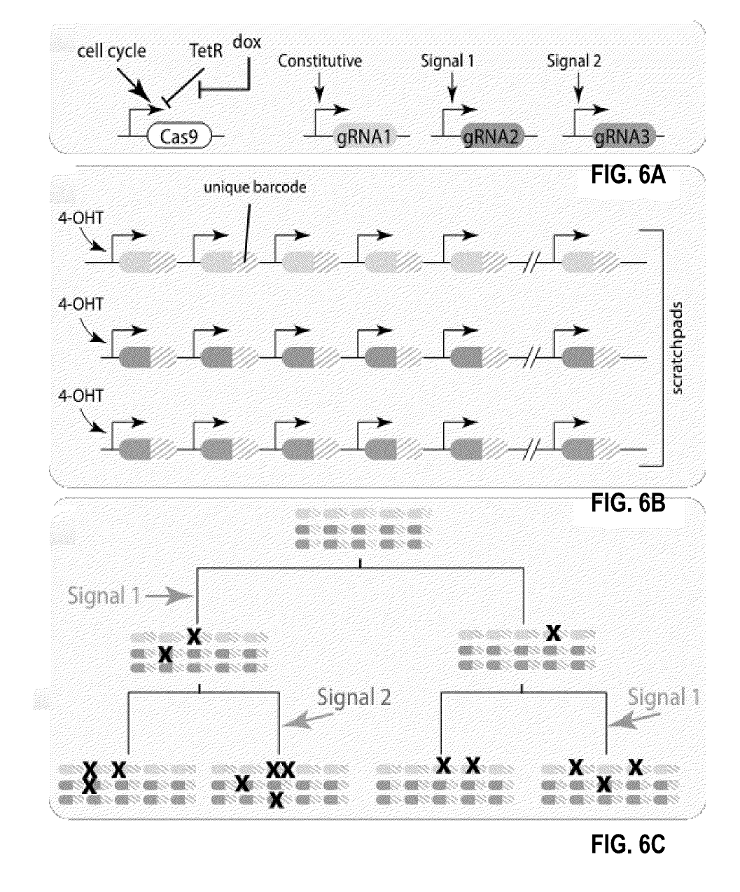

In some embodiments, constitutive and conditional focused mutagenesis systems are coupled. In an exemplary embodiment, a set of gRNAs is activated by a particular constitutive promoter, and is identical with the system discussed previously in connection with event writing. Each additional set will be conditional, being activated by a transcription factor of interest. It will consist of a promoter sensitive to that transcription factor driving a distinct gRNA, which will in turn target a distinct set of barcoded spacers in scratchpad target sites. Reading out of genotypes, as previously described, will be extended to include the additional scratchpads regions. The key idea is that the conditional systems will generate mutations only during intervals when the corresponding gRNA is expressed. By superimposing mutagenic events from the constitutive and signal-dependent gRNAs, one can reconstruct not just the lineage tree, but also the branches in which signaling events occurred (e.g., FIG. 6).

In the exemplary embodiment depicted in FIG. 6, multiple focused mutagenesis systems are used, each of which utilizes a distinct set of gRNAs and corresponds to a genetic scratchpad.

FIGS. 6A through 6C illustrate that event recording can be integrated into the lineage tracking system using an intersectional strategy. FIG. 6A depicts an exemplary design of one potential event recording system. Cas9 is expressed from a cell cycle dependent promoter and a constitutive promoter drives one guide RNA (gRNA1), as above. In addition, two signal-dependent promoters drive distinct gRNAs (e.g., gRNA2 and gRNA3) that target additional corresponding scratchpads (e.g., FIG. 6B). As a result, signaling events that occur during development can be recorded alongside lineage information, as indicated schematically by the mutations (X's) in (FIG. 6C). While mutations associated with the constitutive promoter can occur during any cell cycle, the mutations controlled by signal-dependent promoters can be turned on and off. This way, certain mutations (e.g., those associated with gRNA2 and gRNA3) are induced only in specific cell cycle.

Signaling pathways provide a model system for recording known inputs. In some embodiments, signaling pathways such as BMP, SHH, and Notch will be analyzed by the methods and systems disclosed herein. Such pathways are critical for diverse developmental processes, easy to manipulate with external ligands and pharmacological inhibitors, and in active use in the lab.

In some embodiments, these pathways will be activated or inhibited in mouse embryonic stem cells (mESCs) containing corresponding recording systems utilizing pathway specific sensors incorporating multimerized binding sites for Smad and CSL transcription factors, respectively.

Focused mutagenesis can enable “analog” recording of event intensity. Stronger signaling events are expected to induce higher expression of corresponding gRNAs, which could increase the mutation rate. As a result, the number of mutations accumulated in any given cell cycle could provide an indication not just of whether a transcription factor was active, but also of how strongly activated it was. To work, the mutation rate and number of target sites must be tuned to the dynamic range of the signal-dependent gRNA promoters. To explore this possibility, the relationship between ligand level and number of mutations induced will be systematically measured using the above signal pathways.

The event recording methods and systems disclosed herein can be used to analyze ES differentiation. In some embodiments, the methods and systems can be used to record the activation of master transcription factors that activate specific lineages under conditions of heterogeneous differentiation. In some embodiments, facts determined from gene expression (antibody staining or single-molecule RNA FISH) are correlated with records of transcription factor activation recorded in the scratchpad of the same cell.

As illustrated, the mutation status can be characterized in mammalian cells as well as simpler eukaryotic or even prokaryotic cells. In some embodiments, individual images of a cell population of interest are collected at different time points over a period of time. In some embodiments, continuous video images are collected over a period of time. In some embodiments, the period of time for image collection can cover any duration of time; for example, it can be over two cell cycle generations or longer, three cell cycle generations or longer, four cell cycle generations or longer, five cell cycle generations or longer, six cell cycle generations or longer, seven cell cycle generations or longer, eight cell cycle generations or longer, nine cell cycle generations or longer, 10 cell cycle generations or longer, 12 cell cycle generations or longer, 15 cell cycle generations or longer, 20 cell cycle generations or longer, 30 cell cycle generations or longer, 40 cell cycle generations or longer, 50 cell cycle generations or longer, 75 cell cycle generations or longer, or 100 cell cycle generations or longer.

In one aspect, provided herein are methods and systems for establishing or reconstructing lineage tree for a cellular process or pathway.

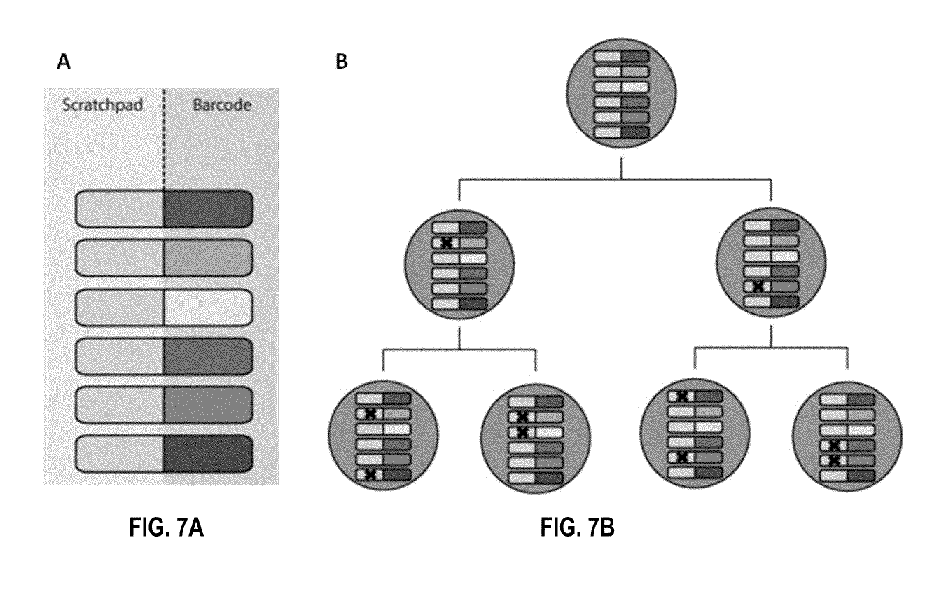

FIGS. 7A and 7B illustrate an exemplary schematic of lineage tree reconstruction based on scratchpad state. FIG. 7A depicts a scratchpad implementation including a region targeted for deletion (colored in gray in the left) and a unique barcode (in rainbow color on the right). FIG. 7B shows a lineage tree that is constructed based on deletions in the scratchpad (labeled as “x” in the figures). In particular, cells with common ancestors can be identified to reconstruct a lineage tree.

The method yields single-cell information and is not restricted to coarse-grained population measurements. It can also provide single-cell-cycle resolution: by adjusting the rate of scratchpad mutation, the time resolution of the technique can be tuned. In particular, mutation rates resulting in at least a few scratchpad mutations per cell cycle enable the reconstruction of lineage trees with single-cell resolution.

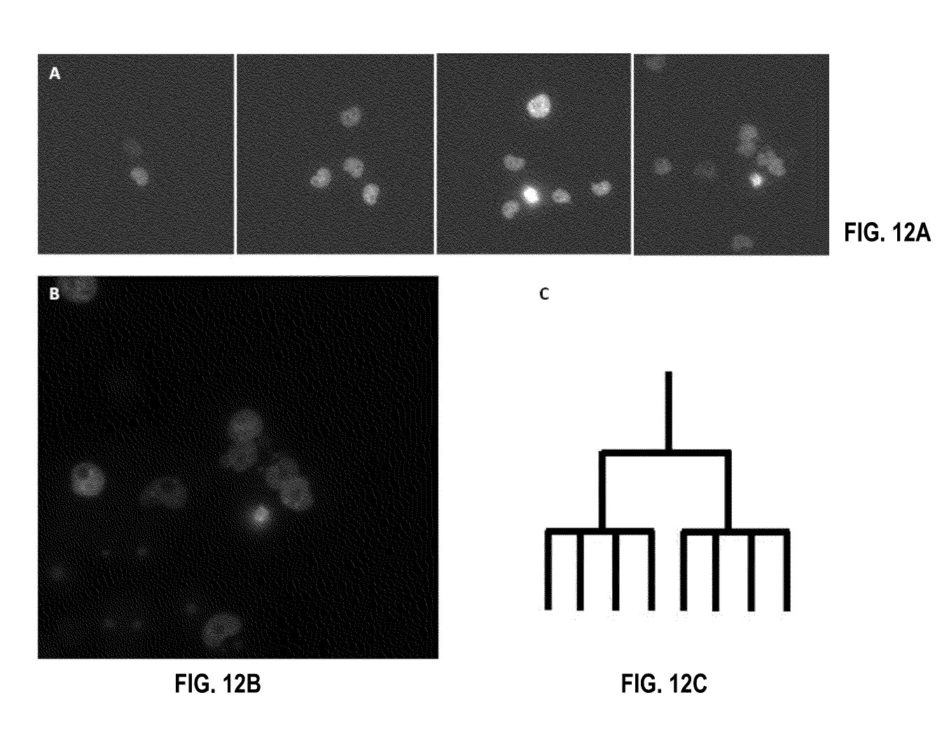

For example, lineage trees can be reconstructed based on inherited changes in each cell's scratchpad state. By reading out the accumulated changes in each cell, we can infer the most likely lineage history of a population of cells (FIGS. 7 and 12). Genomic changes induced by our method are deliberately tuned to occur more frequently than somatic mutations and are in defined locations, which provide improved lineage information (at single-cell resolution) and easier readout, respectively. Moreover, methods relying on somatic mutations are not currently amenable to in situ readout of the lineage information.

The methods and systems disclosed herein are also ideal for applications beyond lineage tracking, including event recording in single cells and tissues. By using multiple variants of scratchpads and writing components, different types of events can be recorded in parallel. And, this method makes it possible to resolve the timing of these events by using lineage tracking principles to map inherited mutations backward in time. Transcriptional, signaling, and other cellular events can be recorded in the genome. Ultimately, this history can be read out and the cell's or tissue's history reconstructed.

Beyond lineage analysis, the system described herein has many additional applications. In some embodiments, the methods and systems disclosed herein can be used to record events leading to tumor genesis or metastasis in tissue and animal models, thereby facilitating understanding of mechanisms underlying tumor formation or migration. In some embodiments, the impact of treatments identified to disrupt tumor genesis or metastasis can be assessed with this same approach.

In some embodiments, the methods and systems disclosed herein are used to identify one or more triggering events for tumor genesis or metastasis. In particular, in some embodiments, it is possible to identify signaling events that give rise to oncogenesis. For example, it is established that gRNA expression can be driven by promoters recognized by RNA polymerase II, therefore, signaling events that give rise to gene expression can also be used to express specific gRNAs. By coupling signal dependent mutagenesis, to a constitutive rate of mutagenesis, as described above, one will be able to identify the series of pathway events that were activated within the cells of a tumor and at what point in the lineage history of the tumor those signaling events occurred.

In some embodiments, the methods and systems disclosed herein are used to identify early activation events in neural development. For example, by coupling gRNA expression to neuronal activity via an early response promoter, such as that driving cFos expression, one will be able to identify the activation history of a given progenitor by coupling the conditional mutagenesis to the constitutive mutagenesis, as described above.

In some embodiments, the methods and systems disclosed herein are used to record changes in membrane potential and activation within post-mitotic neurons and other excitable cell types. As disclosed above, one can achieve conditional gRNA expression with the use of an early response promoter. Optimal CRISPR function may be achieved by balancing gRNA efficiency with gRNA turnover, ensuring that changes in membrane potential of a predetermined strength or duration would be accompanied by mutagenesis. Furthermore, by employing multiple, differentially tuned, gRNAs with unique target recognition, one may be able to record events arising from action potentials of various strengths and durations. Using the same approach, one can condition optimized gRNA expression to genes associated with neurodegeneration, such as Tau or beta amyloid. In this way, events would only be recorded in those neurons overexpressing these genes. Additionally, the magnitude of mutagenesis incorporated into the scratchpad in a given neuron would identify it as the possible origin of the pathogenesis.

In some embodiments, once key events and key players are identified, it is possible to design or screen for target-specific therapeutics.

### REFERENCES

1. Sulston, J. E., Schierenberg, E., White, J. G. & Thomson, J. N. The embryonic cell lineage of the nematode Caenorhabditis elegans. Dev Biol 100, 64-119, (1983).
2. Blanpain, C. & Simons, B. D. Unravelling stem cell dynamics by lineage tracing. Nat Rev Mol Cell Biol 14, 489-502
3. Solek, C. M. & Ekker, M. Cell lineage tracing techniques for the study of brain development and regeneration. Int J Dev Neurosci 30, 560-569.
4. Xu, T. & Rubin, G. M. Analysis of genetic mosaics in developing and adult Drosophila tissues. Development 117, 1223-1237 (1993).
5. Lee, T. & Luo, L. Mosaic analysis with a repressible cell marker for studies of gene function in neuronal morphogenesis. Neuron 22, 451-461, (1999).
6. Tasic, B. et al. Extensions of MADM (mosaic analysis with double markers) in mice. PLoS One 7, e33332.
7. Livet, J. et al. Transgenic strategies for combinatorial expression of fluorescent proteins in the nervous system. Nature 450, 56-62.
8. Levesque, M. J., Ginart, P., Wei, Y. & Raj, A. Visualizing SNVs to quantify allele-specific expression in single cells. Nat Methods 10, 865-867.
9. Chung, K. et al. Structural and molecular interrogation of intact biological systems. Nature 497, 332-337.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### CRISPR System Deletes Portions of Genetic Scratchpads

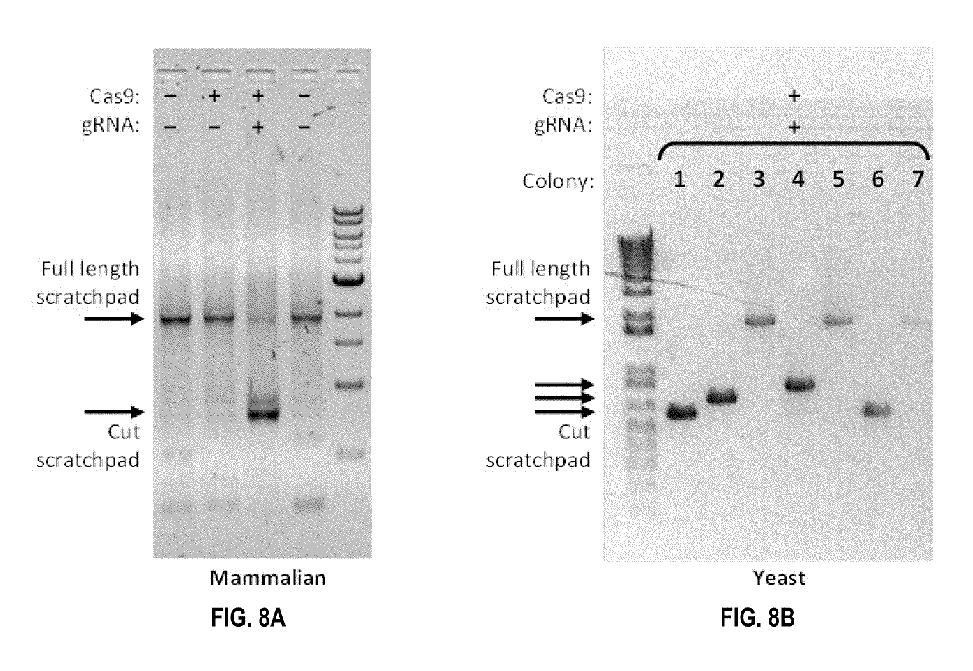

FIGS. 8A and 8B demonstrate that the CRISPR system can write on a genetic scratchpad and results in deletions of portions of sequences of the scratchpad.

FIG. 8A shows the result of bulk PCR of scratchpad in mammalian cells. Scratchpad remains intact in the absence of both gRNA and Cas9, but can be deleted when Cas9 and gRNA are both expressed. A band representing cut scratchpads is clearly visible when both gRNA and Cas9 are present, but absent when either component is missing.

FIG. 8B shows the results of individual yeast clones analysis. Here, efficient removal by the CRISPR system of most repeats of a repetitive scratchpad core is clearly observed, as indicated by multiple bands corresponding to loss of repetitive sequences from a scratchpad core. This writing approach is applicable in many organisms, including mammalian and yeast cells.

### Example 2

### Tuning of CRISPR System

This example illustrates that the cutting efficiency of Cas9 protein in the CRISPR system can be adjusted. As part of this system, Cas9 activity can be tuned through a variety of promoters, mutations, and accessory peptide fusions.

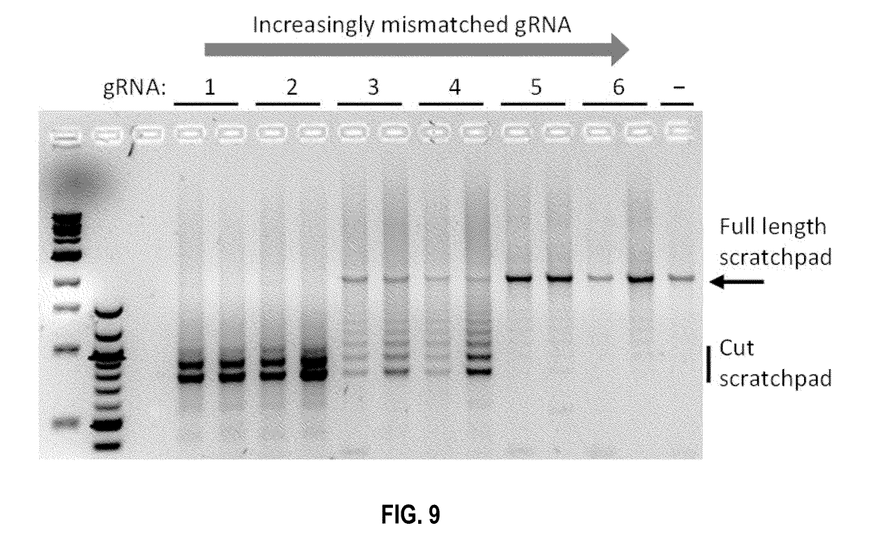

Guide RNAs can also be tuned through the use of mismatched gRNA sequences (FIG. 9), the presence of decoy gRNA, gRNA copy number control, gRNA expression from inducible promoters, and gRNA expression from atypical geometries, such as from introns. Writing can also be achieved via other systems that can alter the DNA scratchpad, including recombinase and integrase enzymes.

As shown in FIG. 9, mismatched gRNAs are one way to tune the rate of scratchpad cutting with the CRISPR system. Mismatched gRNA are not fully complementary to their target site and alter the efficiency of scratchpad cutting. gRNA less complementary to their scratchpad target show reduced (or no) cutting efficiency via bulk PCR.

### Example 3

### In Situ Characterization of Scratchpad and Mutation Status

Our method is ideal for in situ readout of events from individual cells or tissues. By using RNA FISH, we are able to visualize changes in the transcribed DNA that result from our multiple recorded events.

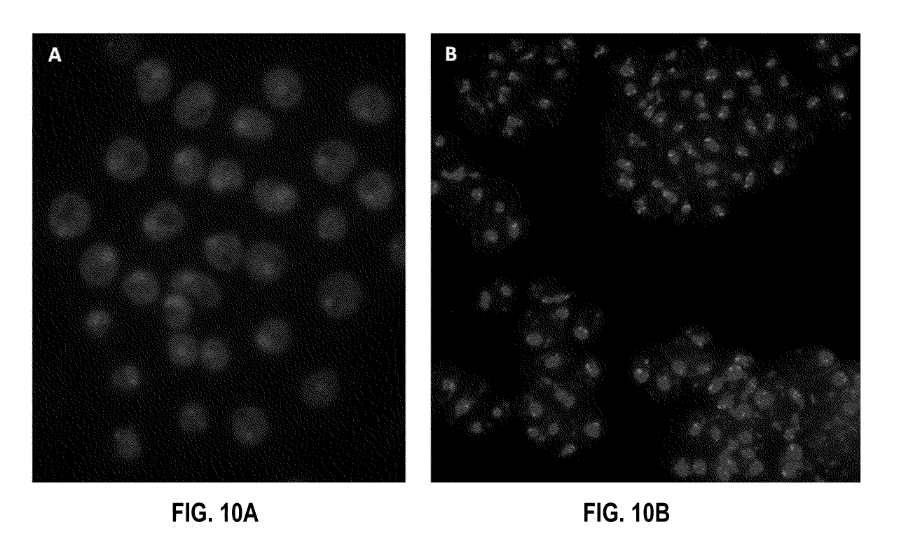

One implementation of this involves transcription of scratchpads from their promoters and subsequent labeling of these nascent transcripts via RNA FISH. The presence or absence (if deletion occurred) of each scratchpad as well as its uniquely identifying downstream barcode region (FIGS. 10 and 11) were visualized.

FIGS. 10A and 10B show scratchpads visualized by FISH in single cells. In FIG. 9A, a colony of mouse embryonic stem cells (red nuclei) that grew from a single cell show RNA FISH images of the scratchpad transcript (blue; seen here as one large dot). In FIG. 9B, yeast cells (blue nuclei) also show scratchpad transcripts (pink) by FISH.

FIGS. 11A and 11B illustrate scratchpad deletion observed by FISH. In both 10A and 10B, in cells lacking gRNA expression, scratchpad transcripts continue to be observed by FISH (blue dots). However, in cells transfected with a strong gRNA (identified by a co-transfection marker (green)), scratchpad transcripts (blue) are no longer present.

### Example 4

### Single Cell Scratchpad Analysis

In this example, single cell scratchpad changes read out by FISH are used to accurately reconstruct of lineage trees.

FIG. 12A shows snapshots from a movie of ES cell colony formation. The bright cell in the top left image underwent three rounds of division, resulting in eight cells. These cells contained scratchpads, Cas9, and gRNA that targeted the scratchpads for deletion over time. FIG. 12B shows the images of the final colony (green cells) by FISH of scratchpad transcripts (blue), which were used to identify cells that retained or lost scratchpads. Four of the eight cells in this colony lost their scratchpads. Based on this information, these four cells most likely underwent a scratchpad deletion event in their common ancestor and are cousins belonging to a subclade of that ancestor.

FIG. 12C shows the schematic of the maximum likelihood lineage tree inferred from FISH observations in these eight cells. The accuracy of this tree can be confirmed here by comparison with the lineage directly observed for these cells in their colony formation movie (A, most frames not shown).

### Example 5

### Sequential Barcoding to Multiplex RNA Detection in Single Cells

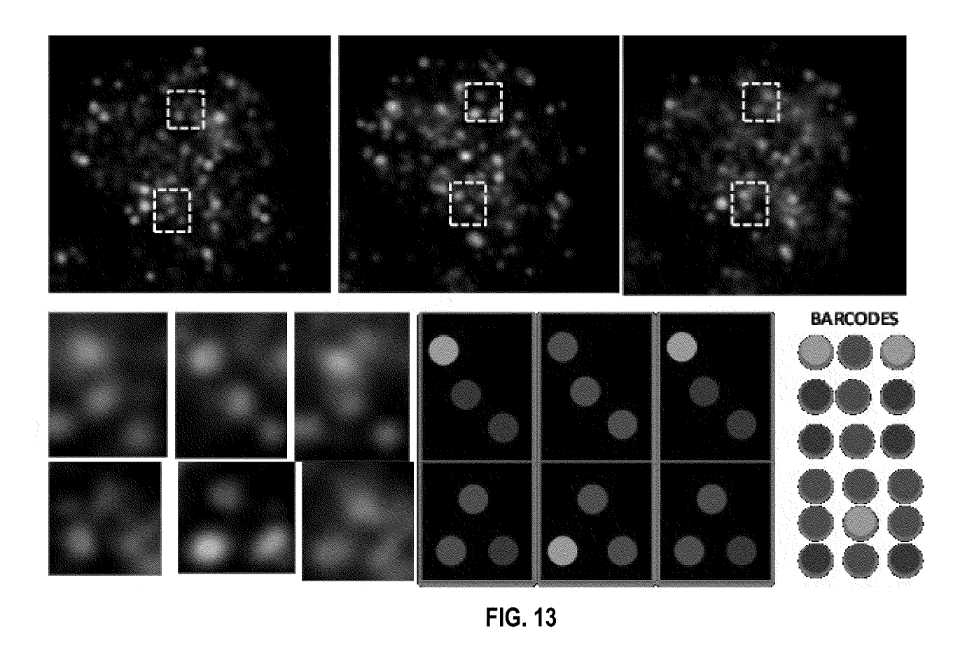

This example includes experimental data demonstrating successful sequential barcoding of transcripts in single cells, as described schematically in FIGS. 4A through 4C. Referring to FIG. 13, each dot corresponds to a distinct mRNA molecule in the cell. Three images (top left to right) show three rounds of hybridization: Hyb1, Hyb2 and Hyb3. Both Hyb1 and Hyb3 used the same labeled probes so dots colocalize, as shown in the lower panels. The lower left panel shows the zoomed in boxed region and the extracted barcodes, represented on the right, demonstrating co-localization of signals. Bottom right panels indicate interpretations of corresponding lower left panels.

### Example 6

### Simulated Recording and Multi-Generation Lineage Reconstruction

This example shows that accurate and robust algorithms can be used to reconstruct the lineage tree from a field of cells with mutagenized recording regions.

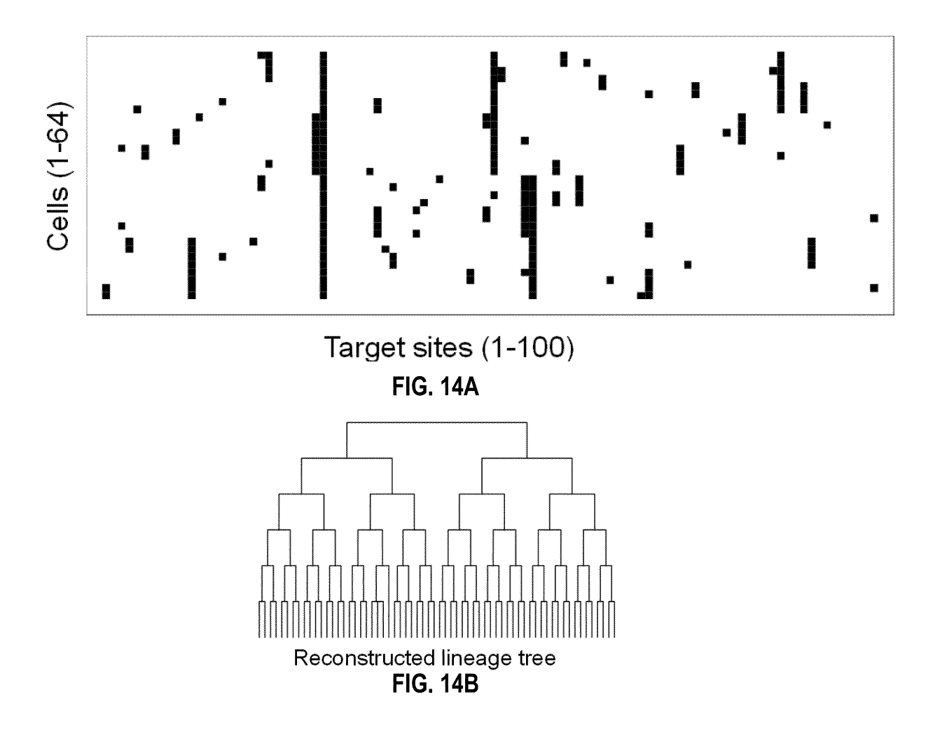

Without the spatial information on cells, computer simulation showed that 100 target sites in the recording region are sufficient to faithfully generate a 10-generation deep lineage tree (FIGS. 14A and 14B). The recording region was readout in situ preserving the spatial organization of cells, it was possible to determine through additional simulations whether this provides an additional level of robustness into the reconstruction process as well as increases the number of generations that can traced with the same number of cutting sites.

FIGS. 14A and 14B shows simulated recording region cut sites and reconstruction for a 6-generation lineage tree. In FIG. 14A, one cell was propagated for 6 generations to generate 64 descendant cells (y-axis). In each generation, a random target site from target sites No. 1-100 was cut per cell (x-axis). The recording region is shown at the end of the 6 generations. Here, a black box indicates that a target site (x axis) is mutated in a given cell, (y axis). In FIG. 14B, based on the data from FIG. 14A, a lineage tree was correctly reconstructed using Manhattan distance and complete linkage models (Mathematica).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for characterizing lineage information or recording molecular events among cells in a cell population, comprising:
introducing, over a time period of multiple cell cycle generations, a plurality of molecular changes in at least one of one or more genetic scratchpads in one or more cells in a cell population,
wherein the cell population comprises cells that have developed for one or more cell cycle generations,
wherein each genetic scratchpad in the one or more genetic scratchpads comprises a polynucleotide sequence and a plurality of target sites within the polynucleotide sequence, and
wherein each of the plurality of molecular changes is associated with a target site among the plurality of target sites;
characterizing, at one or more time points during the time period, a status of molecular changes at each time point for the plurality of target sites in each genetic scratchpad in cells in the cell population, wherein the cells are essentially intact or undisrupted, wherein at least one time point in the one or more time points is two or more cell cycle generations from the beginning of the time period; and
establishing lineage connections or a sequence of molecular changes between cells from different cell cycle generations by comparing statuses of molecular changes of the cells, wherein the molecular changes may represent one or more molecular events.

2. The method of claim 1, wherein said characterizing step further comprises:
applying a set of probes to the cell population,
wherein each probe in the set recognizes and binds to a corresponding target sequence in a target site among the plurality of target sites, and
wherein each probe comprises a label that produces a visible signal upon binding between the probe and its unique target sequence; and
characterizing the of molecular changes status in a plurality of cells in the cell population by detecting the presence or absence of visible signals in the plurality of cells.

3. The method of claim 1, wherein each target site comprises a guide sequence that is recognized by a unique guide molecule, and wherein binding of the unique guide molecule to the guide sequence recruits a molecule that is capable of creating a molecular change at the target site.

4. The method of claim 3, wherein the guide sequence comprises a nucleotide sequence having a length between about 15 nucleic acids to about 80 nucleic acids.

5. The method of claim 3, wherein the guide sequence comprises a nucleotide sequence having a length between about 15 nucleic acids to about 30 nucleic acids.

6. The method of claim 3, wherein the unique guide molecule is a guide RNA (gRNA).

7. The method of claim 3, wherein the molecule is a nuclease, recombinase or integrase.

8. The method of claim 7, wherein the nuclease is Cas9 nuclease

9. The method of claim 1, wherein the multiple time points during the time period cover two or more cell cycle generations.

10. The method of claim 1, wherein the multiple time points during the time period cover three or more cell cycle generations.

11. The method of claim 1, wherein the multiple time points during the time period cover five or more cell cycle generations.

12. The method of claim 1, wherein the plurality of molecular changes comprises a plurality of mutations.

13. The method of claim 12, wherein the plurality of mutations comprises one selected from the group consisting of an insertion mutation, a deletion mutation, a point mutation, multiple point mutations, and combinations thereof.

14. The method of claim 3, wherein each target site further comprises a barcode sequence linked to the guide sequence.

15. The method of claim 14, wherein the barcode sequence comprises a nucleotide sequence having a length between about 400 nucleic acids to about 2,000 nucleic acids.

16. The method of claim 14, wherein the barcode sequence comprises a nucleotide sequence having a length between about 50 nucleic acids to about 200 nucleic acids.

17. The method of claim 1, wherein each target site in a plurality of target sites within at least one genetic scratchpad comprises the same guide sequence that is recognized by a unique guide molecule.

18. The method of claim 1, wherein each target site in a plurality of target sites within at least one genetic scratchpad comprises a different guide sequence that is recognized by a unique and different guide molecule.

19. The method of claim 18, wherein the plurality of target sites within at least one genetic scratchpad comprises one selected from the group consisting of two or more different guide sequences, three or more different guide sequences, five or more different guide sequences, eight or more different guide sequences, 10 or more different guide sequences, 15 or more different guide sequences, 20 or more different guide sequences, and 30 or more different guide sequences.

20. The method of claim 1, wherein the characterizing step further comprises:
applying a set of probes to cells in the cell population, wherein each probe comprises a nucleic acid sequence designed to bind to a target site within the plurality of target site, and wherein each probe is associated with a label that produces a signal upon binding between the probe and its corresponding target site;
characterizing a mutation status at the plurality of target sites based on the absence and presence of signals, wherein absence of a signal indicates a mutation at the target site and the presence of a signal indicates an intact target site, or vice versa.

21. The method of claim 20, wherein the set of probes comprises RNA probes or DNA probes.

22. The method of claim 20, wherein probes in the set of probes are associated with multiple labels that produce different signals.

23. The method of claim 20, wherein each probe of the set of probes is designed to bind to a guide sequence within a target site within the plurality of target site.

24. The method of claim 23, wherein each probe of the set of probes is designed to further bind to a barcode sequence linked to the guide sequence within a target site within the plurality of target site.

25. A system for characterizing lineage information or molecular events among cells in a cell population, comprising:
a housing component for one or more cells in a cell population, wherein a plurality of molecular changes is introduced over a time period of multiple cell cycle generations in at least one of one or more genetic scratchpads in one or more cells in a cell population,
wherein the cell population comprises cells that have developed for one or more cell cycle generations,
wherein each genetic scratchpad in the one or more genetic scratchpads comprises a polynucleotide sequence and a plurality of target sites within the polynucleotide sequence, and
wherein each of the plurality of molecular changes is associated with a target site among the plurality of target sites;
a characterization component, configured to characterize the cell population, at one or more time points during the time period, a status of molecular events at each time point for the plurality of target sites in each genetic scratchpad in cells in the cell population, wherein the cells are essentially intact or undisrupted, wherein at least one time point in the one or more time points is two or more cell cycle generations from the beginning of the time period; and
an analytical component, designed to receive data from the characterization component and establish lineage connections or a sequence of molecular changes between cells from different cell cycle generations by comparing statuses of molecular changes of the cells, wherein the molecular changes may represent one or more molecular events.
